(19) United States
(12) Patent Application Publication
(10) Pub. No.: US 2005/0287159 A1
(43) Pub. Date: Dec. 29, 2005

(54) Treatment methods for eotaxin mediated inflammatory conditons

(76) Inventor: Dimitrios T. Drivas, Scarsdale, NY (US)

Correspondence Address: HOXIE & TSO LLP, 374 MILLBURN AVENUE, SUITE 300 E, MILLBURN, NJ, 07041, US

(21) Appl. No.: 10/509,292

(22) PCT Filed: Mar. 24, 2003
(86) PCT No.: PCT/US03/08970

§ 371 Date: Sep. 23, 2004

Related U.S. Application Data
(60) Provisional application No. 60/367,591, filed on Mar. 25, 2002.

(51) Int. Cl. A61K 48/00; A61K 39/00

(52) U.S. Cl. 424/185.1; 514/44

(57) ABSTRACT

Immunogenic compositions which generate an active immune response in a subject comprising autoantibodies to eotaxim which neutralize its biological activity and treatment methods using such compositions.

### BACKGROUND OF THE INVENTION

Cytokines are peptide messenger molecules that are produced by and act on the cells of the immune system. They are paracrine or autocrine in character and may act systemically if they escape cell binding and spill over to general circulation through the lymph or plasma. While cytokines play a critical role as the chemical messengers of the immune system and are essential to normal immune function, in certain immune system disorders the levels of specific cytokines are abnormal and potentiate the disease state.

In immune system disorders such as atopic conditions, in particular asthma, and in autoimmune diseases, chemokines, a particular class of cytokines, and their subclass interleukins, play an important role. The presence and levels of these chemokines in tissues induce physiological changes, which in individuals suffering from a particular disease are amplified and perpetuated so as to result in a phenotype, which is recognized as the disease state. Chemokines are mediators of the initiation and maintenance of inflammation. Disrupting the chemokine-receptor interactions with neutralizing anti-chemokine antibodies or with chemokine receptor antagonists may diminish or inhibit inflammatory responses. Autoantibodies to chemokines can effectively neutralize chemokines and their signaling and modulatory effects on the immune system and disease. The concept of a therapy for diseases associated with abnormal levels of chemokines through the regulation of a patient's autoantibody levels to the target chemokine may in fact emulate the body's own etiology or regulation of the disease.

The important modulatory role of chemokines in disease has resulted in a number of products in development whose mode of action is to block the binding of the chemokines to their receptors. The majority of these products, some of which are in clinical trials, are based on humanized monoclonal antibodies (“mAbs”), non-antigenic receptor antagonists or soluble receptor molecules or analogues; all of which require many repeat administrations and do not ideally lend themselves to long term therapy or prophylactic treatment. For example humanized anti-TNFalpha mAbs for rheumatoid arthritis and inflammatory bowel disease, and several humanized anti-IL-4, anti-IL-5, anti-IL-8 and anti-IL-9 mAbs for the treatment of asthma are in development. These humanized mAb treatments may have potential for the short term treatment of acute disease states, however, they are not ideally suited for long term maintenance therapy. As a result, therapies which result in an effective harnessing of the patient's own immune system to mount a polyclonal autoantibody based control of the target chemokine levels have been suggested as a means to overcome many of the disadvantages of the products currently in clinical trials (see for example, WO 00/65058 and U.S. Pat. No. 6,093,405).

Cytoline Neutralization

The most prevalent methods of cytokine neutralization under development are by administration of cytoline receptor antagonists, by the administration of humanized monoclonal antibodies against the cytokine or the cytokine receptor, or by the administration of truncated forms of the receptor, which bind to the cytokine and neutralize it. For example, U.S. Pat. Nos. 5,912,136; 5,914,110; 5,959,085; 6,168,791 B1; and 6,171,590 B1 all disclose such methods. Another reported method of neutralization of cytokines is through the use of antisense molecules complementary to the coding sequence of the cytokine gene, the goal of which is to inhibit the expression of the gene.

Cytoline neutralization with autoantibodies generated by active immunization is now considered a promising method of treating pathological conditions (Zagury et al., “Toward a new generation of vaccines: The anti-cytokine therapeutic vaccines”, PNAS, Jul. 3, 2001, Vol. 98, No. 14, 8024-8029, Svenson et al., Journal of Immunological Methods 236 (2000) 1-8, Richard et al., PNAS, Jan. 18, 2000, Vol. 97, No. 2, 767-772. Dalum et al., Nature Biotechnology, Vol. 17, July 1999, 666-669). Vaccines useful for cytokine neutralization can be produced by inactivating the cytokine molecule and rendering it immunogenic, see for example Ciapponi et al., (“Induction of interleukin-6 (IL-6) autoantibodies through vaccination with an engineered IL-6 receptor antagonist.” Nature Biotechnology, Vol. 15, October 1997, pgs. 997-1001) who successfully demonstrated the neutralization of IL-6 after vaccination with an antigenic, non-biologically active, engineered IL-6 receptor antagonist in transgenic mice with high circulating levels of human IL-6. Ciapponi et al., speculate on the advantage of such a vaccination treatment of immune or neoplastic diseases over therapies with monoclonal antibodies (mAbs) or receptor antagonists, which require continuous parenteral delivery. Alternatively, the cytokine can be coupled to an immunogenic carrier to render it immunogenic (see for example Richard et al., PNAS, Jan. 18, 2000; Vol. 97, No. 2, 767-772, U.S. Pat. No. 6,455,504, U.S. Pat. No. 6,420,141, WO 01/43771 and WO 00/64397). The anti-cytokine vaccine approach has been proposed for the treatment of asthma and allergic diseases by controlling the levels of interleukins implicated in these disease states, (see WO 00/65058 and U.S. Pat. No. 6,093,405).

Atopic Conditions: Asthma, Allergy and Allergic Diseases.

Asthma is becoming one of the most important medical problems with about 15 million asthma sufferers in the US alone. The number of asthma sufferers has increased over 50% in the last 10 years with 700,000 victims, mostly children, emerging in the US each year.

While all humans produce a protective immune response to allergens that enter the lungs, some individuals react by producing an overwhelming response of cells producing the allergic immune antibody, IgE, which release substances, including chemokines, that cause asthma attacks. Chronic asthma, in which asthmatic symptoms are exhibited at least twice a week, is currently treated with two types of drugs: (1) a medication that quells inflammation such as a corticosteroid and (2) a rescue drug to open constricted airways and make breathing easier when attacks occur. The current drugs on the market only help in relieving the symptoms of asthma and do not eliminate or suppress the immune response that causes the allergy and subsequently the asthma. In addition, the majority of current medications are either pills which must be taken frequently or must be administered frequently or during an attack with an inhaler. As predominantly steroid-based therapies they may also result in undesirable side effects and decreased efficacy with increased or long-term use. A vaccine type medication, administered only every few months, that suppresses or eliminates the allergic-like response that results-in-asthmatic-attacks is highly desirable.

T-helper cells perform a key function of the immune response. Generally T-helper precursor (Th-p) cells differentiate as part of the immune response into either T-helper 1 (Th-1) or T-helper 2 (Th-2) effector cells each of which have important biological roles. In response to an antigen, which enters the lungs, an individual can either develop a protective Th-1 or an allergic Th-2 response. The Th-2 response results in the production of IgE antibodies and allergy symptoms. Allergic and asthmatic individuals exhibit an overwhelmingly Th-2 response to inhaled allergens associated with elevated levels of IgE.

The airway inflammation in asthma is characterized by an infiltration of the airway wall by Th2 cells, eosinophils and mast cells. Each of these cells contributes to the physiological changes that characterize asthma and each of the cell types produce and are responsive to a limited panel of cytokines.

The differentiation of Th-p cells into either Th-1 or Th-2 cells is mediated by different hormone signaling pathways comprised mostly of different sets of interleukin chemokines. The Th-2 pathway is mediated by IL-4, IL-5, IL-6, IL 10 and IL-13. These interleukins are produced by Th-2 cells and are critical for antibody production and the signaling to other cells involved in the allergic immune response. Eotaxin is another chemokine further down the Th-2 signaling cascade, which regulates eosinophil cells, affects the production of IgE antibodies and is critical to the allergic response.

There is currently a substantial research and development effort in new asthma therapies. These include the chemokine neutralization therapies primarily for IL-4 and IL-5 and other strategies aimed at neutralizing IgE antibodies either by direct blockade with humanized anti-IgE mAbs or possibly a vaccine to immunize against IgE. Other, more conventional allergy vaccine strategies, center on immunization or desensitization with specific peptide allergens, for example, cat dander or ragweed pollen. These more conventional areas of research also continue with efforts on new delivery methods and application of DNA vaccine technology to allergen vaccination, however, allergen-specific strategies do not provide a general therapy for asthma.

There is also a considerable research effort for a general asthma vaccine focused on either generating a nonspecific Th-1 immune response, or shifting a patient's Th-2 response to a Th-1 response by immunizing with known Th-1 antigens or DNA vaccines which result in a Th-1 immune response, (see for example U.S. Pat. No. 6,086,898). The active immunization approaches that have been suggested as therapy for allergy and autoimmune disease have focused on controlling levels of the interleukins IL-4 and IL-5. U.S. Pat. No. 6,093,405 discloses inducing an immune response against IL-4 or IL-5 by actively immunizing with an immunogenic IL-4 or IL-5 cytokine composition in order to treat allergy or autoimmune disease respectively. WO 00/65058 discloses the construction of anti-IL-5 immunogens and their use in a method to down-regulate 15 in a proposed method of controlling asthma and other chronic allergic diseases. The choice of the target chemokine is critical in the treatment of any disease state by active immunization. None of these references suggest a method of controlling asthma by active immunization against eotaxin.

Eotaxin

Eotaxins are eosinophil-specific chemokines, which stimulate eosinophil accumulation or attract eosinophils. Eotaxins induce chemotaxis of eosinophils but do not significantly induce the chemotaxis of neutrophils, monocytes or T-cells. The eotaxins are members of the CC subfamily of chemolines, a class which also includes monocyte chemotactic proteins (MCPs) and macrophage inflammatory proteins (MIPs), see: Van Coillie et al., Cytokine & Growth Factor Reviews, 10 (1999) 61-86; Garcia-Zepeda, et al. (1996) Nat. Med., 2: 449-456.

There are currently at least three molecules classified as eotaxins; the first to be identified, eotaxin-1 and still referred to as eotaxin, (see Kitaura, M. et al., J. Biol. Chem., 1996, 271; 7725-30 and Ponath et al., J. Clin. Invest. 1996, 97: 604-12.) and the later discovered eotaxin-2 and eotaxin-3, (see Conroy et al. Respir Res 2001, 2: 150-156; Guiterrez-Ramos et al. Immunology Today, November 1999, Vol. 20, No. 11, 500-504). Eotaxin binds to and acts through the chemokine receptor 3, CCR3, with relatively high affinity to induce eosinophil recruitment. The structure and peptide sequence and the genes which encode eotaxins are known, and receptor binding has been studied and characterized (see Garcia-Zepeda et al. Nature Medicine, Vol. 2, No. 4, April 1996, 449-456; Ye et al., The Journal of Biological Chemistry, Vol. 275, No. 35, Sep. 1, 2000 27250-27257; Mayer and Stone, The Journal of Biological Chemistry, Vol. 276, No. 17, Apr. 27, 2001, 13911-13916.

Eosinophils are one of the principle components of the body's Th-2-type immune defense to helmitic parasitic infections and accumulate in the blood and tissues of infected individuals. The eosinophils contain granules of cationic proteins, which upon degranulation are released into the cell's environment and damage the invading helminth. Atopic conditions such as asthma and chronic allergic diseases are characterized by a predominant Th-2 type immune response to allergic non-helmitic stimuli. Inflammation of the lung in patients with asthma and chronic allergic diseases is characterized by infiltration and accumulation in the lung and in particular of the bronchial mucosa of eosinophils. In these conditions in the absence of helmitic infection the release of the eosinophil's cationic proteins upon degranulation damages the surrounding cells. As a result, eotaxin has been recognized as a potential target for the treatment and prevention of atopic conditions and in particular the therapy of asthma and allergic disease. U.S. Pat. Nos. 5,993,814 and 6,031,080 and PCT publications WO 95/07985, WO 97100960, WO 97/12914, and WO 99/10534 suggest the use in therapy of various eotaxin agonists and antagonists including antibodies against eotaxin. WO 01/66754 discloses the production and use of anti-eotaxin human antibodies CAT 212 and 213 and fragments thereof for the treatment of eotaxin mediated conditions in a passive immunization regimen.

The receptor on which eotaxin acts, the CC, CKR3 or CXCR3 receptor, has been characterized, (see WO 97/41154 and U.S. Pat. No. 6,171,590 B1) and agonists and antagonists of this receptor have also been suggested for therapy (see U.S. Pat. No. 6,271,347). U.S. Pat. No. 6,171,590 B1 suggests that immunogenic oligopeptides derived from the receptor can be used in active immunization against the receptor for a therapeutic effect. None of the publications or patents referred to above, however, suggest the active immunization against eotaxin itself as a therapeutic method or disclose immunogenic compositions useful for such active immunotherapy.

### SUMMARY OF THE INVENTION

The invention concerns vaccine products targeting the cytokine eotaxin and their use to treat animal subjects, including humans, for inflammatory conditions that result from eotaxin mediated eosinophil accumulation such as asthma and allergic diseases and other atopic conditions. The vaccine or immunogenic products employ different immunogen types and delivery methods and can be used alone or in combination with other therapeutic agents.

The antigenic peptide-based products may be formulated using a modified eotaxin that is rendered inactive and immunogenic. In certain embodiments the immunogens comprise at least one eotaxin receptor antagonist or agonist, or an eotaxin-derived epitope conjugated to an immunogenic carrier. Immunogens derived from eotaxin or eotaxin mimetics can be constructed using methods well known in the art and used to elicit an immune response in laboratory animals such as mice or rabbits. The resulting antibodies can be screened for their ability to neutralize eotaxin binding to its receptor in vitro as a prelude to selecting the most appropriate epitope for clinical development. The DNA based products comprise DNA vaccine products that encode and result in the production in the treated subject of the antigenic peptide products, which will elicit an immune response against eotaxin in the immunized subject. The design of any particular product depends on the target tissue in which the primary immune response is sought and the type of immune response which will be primarily generated.

The immunogenic compositions generate an autoantibody response in the subject or patient to the eotaxin at a level, which is sufficient to immunoneutralize eotaxin and thus down regulate the eotaxin activity and result in a reduction of eotaxin mediated eosinophil accumulation so as to ameliorate the inflammatory condition. The immunogens may include: combination peptide immunogens comprising portions of the eotaxin sequence or mimetics coupled to an immunogenic carrier or DNA vaccines encoding such combination peptide immunogens.

In one embodiment, a specific peptide sequence, which is an epitope present on eotaxin or an immunomimic thereof, is coupled to an immunogenic carrier to produce the anti-eotaxin immunogen. The immunogen is administered in a suitable formulation such as water in oil emulsion to the subject by injection in order to elicit a humoral immune response that produces antibodies neutralizing towards eotaxin. The immune response may be maintained for a sustained period by the administration of booster doses of the anti-eotaxin immunogen. Suitable immunogenic carriers may include proteins or protein toxoids such as Diphtheria toxoid (DI) or Tetanus toxoid (TT), Keyhole limpet hemocyanin (KLH), Influenza virus haemaglutinin, etc. A specific peptide sequence is coupled to the immunogenic carrier via conjugation with bifunctional cross-linking agents. Alternatively, the specific peptide may be synthesized in tandem with a suitable T-cell epitope sequence(s) as a synthetic heterofunctional immunogenic peptide carrying the specific B-cell epitope and T-cell epitopes to elicit a sustained immunogenic response to the desired eotaxin target fragment. The specific peptide sequence may be expressed in tandem with T-cell epitopes as a fusion protein using a suitable plasmid vector for expression of the protein in vitro using methods known in the art or in vivo after vaccination with the vector DNA.

The invention also concerns methods for the treatment of conditions associated with cotaxin mediated eosinophil accumulation comprising the active immunization of a subject against eotaxin with the immunogenic compositions of the invention. The inventive treatment methods include therapies, which involve the treatment of the subject with other pharmaceutical agents in addition to the active immunization using the anti-eotaxin vaccine.

### DETAILED DESCRIPTION OF TH INVENTION

The invention provides compositions, which are useful for the treatment of conditions characterized by eotaxin, mediated eosinophil accumulation. These atopic conditions of which asthma and chronic allergic diseases are the most prevalent include atopic skin conditions such as psoriasis and other conditions such as eosinophilic ulcerative colitis. In each of these conditions eosinophils accumulate in the affected tissue to a large extent through eotaxin induced eosinophil recruitment. The chronic presence of elevated levels of eosinophils in the affected tissues results in significant tissue damage which over time progresses and may become irreversible. The prior art therapies, which are being pursued for the mediation of eotaxin's effects, are directed to blocking the action of the chemokine on the CCR3 receptor with small molecule antagonists, passive immunization with human or humanized monoclonal antibodies to reduce eotaxin levels, or passive or active immunization against the CCR3 receptor itself. The small molecule and passive immunization approaches require repeat administration and suffer from the standpoint of patient compliance. Furthermore, the induction of neutralizing antibodies to the administered mABs as a result of repeat therapy can seriously compromise the effectiveness of passive immunotherapy with mAbs for long term treatment of a chronic disease (Adair, F., Drug Discovery World, Summer 2002 pp 53-59). On the other hand active immunization against the CCR3 receptor itself may interfere with the binding of other CC chemokines to the receptor and thus may have unforeseen and unintended biological consequences.

The present invention overcomes the shortcomings of the prior art by providing immunogenic compositions which are useful for active immunization and which can induce a sustained immune response against eotaxin itself in an animal subject including human subjects. The autoantibodies induced in the subject against eotaxin preferably do not strongly cross-react with other CC chemokines such as MIPs, MCPs, eotaxin-2, or eotaxin-3. Various immunogens according to the invention may be produced and analyzed in the appropriate animal models for the disease or inflammatory condition of interest in order to select the specific immunogen, which is optimal for the treatment of the particular condition. Suitable animal models for asthma and other allergic disorders are well known in the art (see Humbles et al., J. Exp. Med., Vol. 186, No. 4, Aug. 18, 1997, 601-612, Corry et al., J. Exp. Med., Vol. 183, January 1996, 109-117, Foster et al., J. Exp. Med., Vol. 183, January 1996, 195-201, Lukacs, et al., Am. J. Respir. Cell Mol. Bio., Vol. 10, 526-532, 1994).

The inventive immunogens should induce an immune response in the subject comprising antibodies of a sufficiently high specificity and binding affinity to eotaxin so as to be able to neutralize or modulate the biological activity of eotaxin. In addition the titer of anti-eotaxin antibodies induced should be sufficient to result in the lowering of elevated levels of eotaxin in the subject and a reduction in the recruitment of eosinophils to the tissues which are affected by the atopic condition. By actively immunizing the subject or patient with the anti-eotaxin immunogenic compositions of the invention, a level of autoantibodies, which react with eotaxin, are maintained in the subject, which may prevent or ameliorate eosinophil recruitment during an allergic reaction. This anti-eotaxin autoantibody level can be maintained with booster administration of the inventive immunogenic compositions and thus should provide superior protection for management of the chronic disease state compared to that afforded by small molecule eotaxin antagonists or passive anti-eotaxin immunization which will exhibit considerable fluctuation in levels of the therapeutic agent, and suffer from less favorable patient compliance. In addition passive immunization of mAbs is subject to development of neutralizing antibodies to the targeted-mAbs upon repeat dosing limiting their effectiveness for long term treatment.

Mature human eotaxin is derived from a 97 amino acid precursor protein which contains a 23 amino acid hydrophilic amino terminal sequence which is cleaved off to leave the mature protein of 74 amino acids and approximate molecular weight of 8.4 kDa (see, U.S. Pat. No. 6,403,782, WO 99/10534, WO 97/00960, Ye et al., Journal of Biological Chemistry, Vol. 275, No. 35, Sep. 1, 2000, 27250-27257, Garcia-Zepeda et al., Nature Medicine, Vol. 2; NO. 4; April 1996, 449-456, Ponath et al., J. Clin. Invest., Vol. 97, No. 3, February 1996, 604-612, Mayer et al., Journal Biological Chemistry, Vol. 278, No. 17, Apr. 27, 2001, 13911-13916). The amino acid sequence of mature human eotaxin (SEQ ID NO 1) is as follows (using the one letter code for each amino acid residue):
GPASVPTTCC¹⁰ FNLANRKIPL²⁰ QRLESYRRIT³⁰ SGKCPQKAVI⁴⁰
FKTKLAKDIC⁵⁰ ADPKKKWVQD⁶⁰ SMKYLDQKSP⁷⁰ TPKP⁷⁴

Eotaxin is not normally immunogenic. The peptide itself or fragments of the peptide corresponding to epitopes of interest can be rendered immunogenic by methods well known in the art. One method that may be used is to produce inactive eotaxins or inactive eotaxin fragments which have lost eotaxin biological activity but which are in an immunogenic form and can elicit anti-eotaxin neutralizing antibodies in an animal or human subject. A number of chemical, physical and immunological treatments are known which may be useful in producing inactive but immunogenic eotaxin or fragments thereof, (see for example: U.S. Pat. No. 6,093,405; Zagury et al., PNAS, Jul. 3, 2001, Vol. 98, No. 14, 8024-8029; Gringeri et al., Journal of Acquired Immune Deficiency Syndrome and Human Retrovirology, Vol. 20, No. 4, Apr. 1, 1999; Ciapponi et al., Nature Biotechnology, October 1997, Vol. 15, 997-1001; Raaberg et al., Pediatric Research, Vol. 37, No. 2, 1995, 169-174; Raaberg et al., Pediatric Research, Vol. 37; No. 2, 1995, 175-181; Gringeri et al., Journal of Acquired Immune Deficiency Syndromes, Vol. 7, No. 7, 1994, 978-988; Zagury et al., Journal of Acquired Immune Deficiency Syndromes, Vol. 5, No. 7, 1992, 676-681).

The eotaxin or fragments thereof can also be rendered immunogenic and otherwise biologically inactive by coupling the peptide or fragment to an immunogenic carrier protein or protein toxoid such as Diptheria Toxoid (DT), Tetanus Toxoid (TI), Keyhole Limpet Hemocyanin (KLH), BCG, OVA or others, by well known methods (see for example: U.S. Pat. Nos. 6,217,881; 6,132,720; 5,891,992; 5,609,870; 5,607,676; 5,468,494; 5,023,077; and 4,201,770 and Richard et al., PNAS, Jan. 18, 2000, Vol. 97, No. 2, 767-772; Svenson et al., Journal of Immunological Methods, 236 (2000), 1-8; Dalum et al., Nature Biotechnology, Vol. 17, July 1999, 666-669; Gonzalez et al., Annals of Oncology, 9: 431-435, 1998; and Dalum et al., The Journal of Immunology, 1996, 157: 4796-4804). Alternatively, modified eotaxin variants or forms which are inactive but immunogenic may be produced by introducing T helper epitopes in tandem to the eotaxin sequence by using the methods disclosed in WO 00/65058 and WO 95/05849.

Botaxin Fragment Conjugate Immunogens

One embodiment of the invention concerns conjugate immunogens which comprise eotaxin peptide fragments corresponding to desired epitopes on the eotaxin molecule which are conjugated to an immunogenic protein carrier such as DT or TT thereby providing promiscuous T-cell epitopes and enabling the immune memory for prolonged antibody response. Such immunogens may be administered to human or animal subjects to develop an active humoral immune response to eotaxin. One embodiment of the invention comprises the entire human eotaxin molecule conjugated to an immunogenic carrier protein such as DT to render it immunogenic. Other embodiments of the invention comprise shorter eotaxin peptide fragments conjugated to an immunogenic carrier protein. The conjugates may be constructed using peptides of approximately between 4 and 50 amino acid residues that comprise an epitope or epitopes, which will induce antibodies in the subject which will cross-react with epitopes present on the eotaxin molecules existing in the subject. The peptide or peptides comprising the epitopes are then conjugated to the protein carrier in a range of peptide to carrier protein molar ratios. The peptide may be conjugated directly to the immunogenic protein or may incorporate a peptide spacer sequence to extend the desired epitope from the carrier molecule in order to enhance its presentation to antigen presenting cells and thereby the immunogenicity of the desired epitope. The conjugation of the peptide to the carrier is accomplished using cross-linking agents, either homobifunctional or heterobifunctional, to attach the desired epitope-containing peptide to the carrier protein. The choice of bifunctional cross-linking agent will depend upon the availability of functional moieties on the peptide. The chemistry for these coupling methods is well known in the art and is set forth in the disclosure of U.S. Pat. Nos. 6,132,720; 5,609,870; and 5,468,494, and Chemistry of Protein Conjugation and Cross-linking, S. S. Wong (1991) CRC Press, Inc.

Alternatively, immunogenic eotaxin peptides may be constructed by synthetic peptide chemistry so as to produce in tandem the selected eotaxin epitope fragment with a T-cell epitope or epitopes, thereby presenting the selected B-cell epitope (derived from eotaxin) with a T-cell epitope to provide for a sustained immune response in the immunized subject.

Immunogenic eotaxin peptides may also be constructed by recombinant DNA technology to produce a plasmid vector in which the desired eotaxin fragment is encoded in tandem with the requisite DNA sequence for T-cell epitope(s), thereby producing a fusion protein comprising the selected B-cell epitope (derived from eotaxin) with a T-cell epitope(s). The fusion protein can be expressed in vitro using cell culture/fermentation techniques or can be used as a DNA vaccine providing for an eotaxin-specific and sustained immune response in the immunized subject.

Referring to the mature human eotaxin sequence, in certain embodiments of the invention peptide fragments from amino acid residue 145 from the amino terminal end of the molecule and fragments from residue 54 to 74, which constitute the carboxyl terminal end are useful in constructing the immunogen conjugates of the invention. The immunogenic conjugate may comprise one or more different eotaxin epitopes that may be present on the same peptide fragment or on different peptide fragments conjugated to the same immunogenic carrier. The conjugate immunogens of the invention may be formulated with adjuvants or other immunostimulatory agents in a pharmaceutically acceptable vehicle with components and using methods well known in the art, (see: Vaccine Design, The Subunit and Adjuvant Approach, (1995) Powell and Newman Eds., Plenum Press (New York), Aucouturier et al., Vaccine 19 (2001) 2666-2672).

The immunogens of the invention may be administered in various dosages in the ranges of approximately micrograms to milligrams of immunogen per administration per subject and by a number of different routes of administration (intranasal, oral, intramuscular or subcutaneous injection, etc.), which may depend on the formulation and the desired immune response (mucosal, systemic, tissue specific, etc.). The peptide conjugate vaccines of the invention may be administered in an appropriate formulation by intramuscular injection in dosages of approximately 0.1 micrograms to 10 milligrams of peptide conjugate per administration. The dosage may need to be adjusted by the administering physician based on the condition being treated and the responsiveness of the patient to the immunogen it being understood that the goal of the treatment is to elicit antibodies which neutralize the biological activity of eotaxin. The dosage regimen per patient may vary depending on the eotaxin concentration levels in the affected tissues of the patient and the anti-eotaxin antibody titers elicited in the patient in response to the immunogen. The immunogens of the invention may also be administered to patients pursuant to a booster regimen in order to maintain an active immune response to eotaxin.

The anti-eotaxin immunogens may also be administered in treatment regimens with other pharmaceuticals or anti-inflammatory agents. For example in the case of asthma or atopic chronic allergic disorders the patient may be actively immunized with an anti-eotaxin vaccine of the invention so as to control and down-regulate eotaxin levels and the accumulation of eosinophils in the affected tissues while at the same time a rescue medication or anti-asthma or anti-allergy agent is administered in response to an acute attack brought on for example by an overwhelming allergic stimulus. Such additional agents useful in combination treatments may include corticosteroids, cromoglyate, anti-inflammatories, COX-2 inhibitors, leukotriene (receptor) antagonists, xanthines, antihistamines and bronchodilators.

Some eotaxin peptide fragments useful in the construction of the immunogens of the invention are as follows: GPASVP (SEQ ID NO 2); GPASVPT (SEQ ID NO 3); GPASVPTT (SEQ ID NO 4); GPASVPTTC (SEQ ID NO 5); GPASVPTTCC (SEQ ID NO 6), GPASVPTTCCF (SEQ ID NO 7); GPASVPTTCCFN (SEQ ID NO 8); GPASVPTTCCFNL (SEQ ID NO 9); GPASVPTTCCFNLA (SEQ ID NO 10); GPASVPTTCCFNLAN (SEQ ID NO 11); GPASVPTTCCFNLANR (SEQ ID NO 12); GPASVPTTCCFNLANRK (SEQ ID NO 13); GPASVPTTCCFNLANRKI (SEQ ID NO 14); GPASVPTTCCFNLANRKIP (SEQ ID NO 15); GPASVPTTCCFNLANRKIPL (SEQ ID NO 16); GPASVPrrCCFNLANRKIPLQ (SEQ ID NO 17); FNLANR (SEQ ID NO 18); FNLANRK (SEQ ID NO 19); FNLANRKI (SEQ ID NO 20); FNLANRKIP (SEQ ID NO 21); FNLANRKIPL (SEQ ID NO 22); KKKWVQDSMKYLDQKSPTPKP (SEQ ID NO 23); KKWVQDSMKYLDQKSPTPKP (SEQ ID NO 24); KWVQDSMKYLDQKSPTPKP (SEQ ID NO 25); WVQDSMKYLDQKSPTPKP (SEQ ID NO 26); VQDSMKYLDQKSPTPKP (SEQ ID NO 27); QDSMKYLDQKSPTPKP (SEQ ID NO 28); DSMKYLDQKSPTPKP (SEQ ID NO 29); SMKYLDQKSPTPKP (SEQ ID NO 30); MKYLDQKSPTPKP (SEQ ID NO 31); KYLDQKSPTPKP (SEQ ID NO 32); YLDQKSPTPKP (SEQ ID NO 33); LDQKSPTPKP (SEQ ID NO 34); DQKSPTPKP (SEQ ID NO 35); QKSPTPKP (SEQ ID NO 36); KSPTPKTP (SEQ ID NO 37) and SPTPKP (SEQ ID NO 38). The peptides may be produced by synthetic or recombinant means, which are well known in the art. One skilled in the art will also understand that the amino acid sequence of any peptide fragment may be modified so as to increase its immunogenicity or in order to impart or enhance some other property of the fragment while maintaining its ability to contribute to the induction of an immune response to eotaxin in the subject Such modifications could be made for example by derivatizing an amino acid residue or by substitution of a particular amino acid for another or by some other method known in the art

Peptidomimetics or immunomimics, which do not exhibit eotaxin biological activity in the particular animal subject, may also be used to construct the conjugate immunogens. The peptidomimetics may not in and of themselves be immunogenic but may be rendered immunogenic by coupling to an immunogenic peptide. In certain embodiments the peptidomimetics may be derived from other mammalian eotaxin molecules such as mouse or guinea pig eotaxin (see U.S. Pat. Nos. 6,031,080 and 5,993,814).

The eotaxin peptide fragments or immunomimics may be conjugated directly to the immunogenic protein carrier or alternatively may incorporate a peptide spacer sequence to extend the desired epitope from the carrier molecule in order to enhance its presentation to antigen presenting cells and thereby the immunogenicity of the desired epitope. A variety of peptide spacers may be used. U.S. Pat. Nos. 5,609,870 and 5,468,494 disclose peptide spacers and methods of conjugating the spacers to peptides of interest and in turn immunogenic protein carriers such as DT or TT which may be useful in constructing the conjugate immunogens of the invention. The peptide spacers; SSPPPPC (SEQ ID NO 39), RPPPPC (SEQ ID NO 40) and LPPPPC (SEQ ID NO 41) may be used for the eotaxin peptide fragments of the invention. The spacer peptides may be incorporated at either the amino terminal or carboxyl terminal end of the eotaxin peptide fragment to produce the peptides which are coupled to the immunogenic protein such as for example:

| | |
|---|---|
| SSPPPPCKKKWVQDSMKYLDQKSPTPKP, | (SEQ ID NO 42) |
| KKKWVQDSMKYLDQKSPTPKPSSPPPPC, | (SEQ ID NO 43) |
| CPPPPSSKKKWVQDSMKYLDQKSPTPKP, | (SEQ ID NO 44) |
| KKKWVQDSMKYLDQKSPTPKPCPPPPSS, | (SEQ ID NO 45) |
| GPASVPTTCCFNLANRKIPLSSPPPPC, | (SEQ ID NO 46) |
| SSPPPPCGPASVPTTCCFNLANRKIPL, | (SEQ ID NO 47) |
| CPPPPSSGPASVPTTCCFNLANRKIPL, | (SEQ ID NO 48) |
| and | |
| GPASVPTTCCFNLANRKIPLSSPPPPC. | (SEQ ID NO 49) |

Typically, the spacer sequences are incorporated into specific eotaxin sequences by synthetic peptide chemistry during preparation of the epitope-containing peptide fragments. In particular, eotaxin fragments which contain sequences with number hydrophilic sequences and which are more likely presented at the surface of the molecule are particularly useful for the invention. These include for example:

| | |
|---|---|
| SGKCPQKAVISSPPPPC, | (SEQ ID NO 50 |
| CPPPPSSSGKCPQKAVI, | (SEQ ID NO 51) |
| FKTKLAKDICSSPPPPC, | (SEQ ID NO 52) |
| CPPPPSSFKTKLAKDIC, | (SEQ ID NO 53) |
| ADPKKKWVQDSSPPPPC, | (SEQ ID NO 54) |
| and | |
| CPPPPSSADPKKKWVQD. | (SEQ ID NO 55) |

In one embodiment, to facilitate the conjugation of the specific eotaxin fragments to carrier proteins such as DT and TT using cross-linking agents it is desirable to eliminate cysteine residues, from the natural sequence by substitution with threonine residues. Thereby, the hydrodynamic quality of the peptide fragment is retained whilst eliminating potentially detrimental side reactions during the cross-linking step. Examples include: SGKTPQKAVISSPPPPC (SEQ ID NO 56), CPPPPSSSGKTPQKAVI (SEQ ID NO 57), FKTKLAKDITSSPPPPC (SEQ ID NO 58), CPPPPSSFKTKLAKDIT (SEQ ID NO 59), ADPKKKWVQDSSPPPPC (SEQ ID NO 60), and CPPPPSSADPKKKWVQD (SEQ ID NO 61).

The anti-eotaxin immunogens may comprise one peptide fragment conjugated to the immunogenic carrier, for example one or more copies of a peptide fragment of the sequence GPASVPTTCCFNLANRKIPL (SEQ ID NO 16) conjugated to DT. In other embodiments, two or more different peptide fragments may be conjugated to the same immunogenic carrier so as to induce an active immune response in the subject with antibodies directed to two or more epitopes on eotaxin. Such an immunogen may for example comprise multiple copies of each of the peptide sequences GPASVPTTCCFNLANRKIPL (SEQ ID NO 16) and KKKWVQDSMKYLDQKSPTPKP (SEQ ID NO 23) conjugated to DT.

In another embodiment a formulation may be prepared using two or more different peptide immunogenic carrier conjugates, so as to induce an active immune response in the subject with antibodies directed to two or more epitopes on eotaxin. For example the composition administered will be a mixture co-formulation of two different immunogen constructs, the first comprising one specific epitope conjugated to the carrier with the second construct comprising a second distinct epitope conjugated to other molecules of the carrier. Such an immunogen formulation may for example comprise multiple copies of each of the peptide sequence DT conjugates GPASVPTTCCFNLANRKIPL (SEQ ID NO 16) conjugated to DT; and KKKWVQDSMKYLDQKSPTPKP (SEQ ID NO 23) conjugated to DT.

The methods of conjugating the peptide to the immunogenic protein carrier are well known in the art. For example the threonine-substituted eotaxin epitope fragments: SGKTPQKAVISSPPPPC (SEQ ID NO 56), FKTKLAKDITSSPPPPC (SEQ ID NO 58), ADPKKKWVQDSSPPPPC (SEQ ID NO 60), may be conjugated to DT or TT carrier proteins via heterobifunctional cross-linking agents. One or more of these eotaxin fragments is cross-linked to DT or TT by reaction with a heterobifunctional cross-linking agent such as N-(epsilon-Maleimidocaproyloxy)-succinimide ester (EMCS) or its water-soluble analogue sulfo-EMCS. In this embodiment, DT or TT are first reacted with the heterobifunctional cross-linking agent via the succinimidyl ester at amino groups on the toxoid. This reaction is preferably accomplished at pH 6.5±0.3 over approximately 1 to 3 hours at room temperature. A ratio of maleimidyl groups to carrier protein of, for example 5:1, 10:1, 15:1 is achieved by reacting the toxoid (DT or other carrier protein) with an appropriate excess of the cross-linking agent. The actual mole excess of cross-linker is determined by titration. The moles of maleimide incorporated per mole of toxoid during titration can be determined by subsequent reaction of the maleimidyl-toxoid with a sulfhydryl compound such as cysteine or beta-mercaptoethanol. The amount of sulfydryl compound reacted with the maleimidyl-toxoid is most readily determined indirectly by reaction of residual sulfhydryl compound with bis-dithio-nitrobenzoate. After removal of excess cross-linking agent by either diafiltartion or gel permeation chromatography the maleimidyl-toxoid is reacted via its terminal sulfhydryl group with a 1:1 mole excess of eotaxin-spacer peptide over maleimidyl moieties of the activated-toxoid. This conjugation of the peptide to the toxoid is preferably accomplished at pH 6.0±0.3 over approximately 3 to 6 hours at room temperature. Alternatively, this conjugation reaction of the peptide to the maleimidyl-toxoid can be accomplished by overnight reaction at room temperature. It may be preferable to conduct the conjugation reactions with cross-linking agents containing maleimide groups in a vessel. After reaction with peptide the excess of peptide is removed by either diafiltration or gel permeation chromatography into phosphate buffered saline pH 7.2±0.2.

The eotaxin-toxoid conjugates can be prepared as single entities suitable for formulation individually or for commingling to provide two or more epitope-specific conjugates in a final formulation. Alternatively, the coupling of two or more eotaxin-specific peptides can be coupled via their terminal sulfhydryl groups to a single maleimidyl-toxoid preparation. This is accomplished by reacting the maleimidyl-toxoid with a mixture of the eotaxin-specific peptides at a combined 1:1 mole excess over the available maleimidyl moieties of the activated-toxoid. Thereby, a single toxoid conjugate carrying multiple eotaxin-specific epitopes is accomplished.

In certain embodiments an immune response to a multiple epitopes on eotaxin is may be induced in the immunized subject to result in a potentially synergistic binding and neutralization of the target eotaxin. Appropriate epitope sequences are selected from those sequences sufficiently removed from one another on the target molecule as to reduce the likelihood for interference between the binding of antibodies to their specific eotaxin epitopes. One embodiment of such a combination of eotaxin epitopes includes the following eotaxin and eotaxin analogue sequences:

| | |
|---|---|
| SGKTPQKAVISSPPPPC | (SEQ ID NO 56) |
| or | |
| CPPPPSSSGKTPQKAVI | (SEQ ID NO 57) |
| in combination with | |
| ADPKKKWVQDSSPPPPC | (SEQ ID NO 60) |
| or | |
| CPPPPSSADPKKKWVQD. | (SEQ ID NO 61) |

Formulations suitable for immunogenic presentation of the eotaxin-specific toxoid conjugates include, but are not limited to, adsorption to aluminum or alhydrogels, inclusion within liposomes, microsomes or similar microspheres, including microparticulates and nanoparticulates, oil-in-water or water-in-oil emulsions, including multiphasic emulsions, and microemulsions. Other potentially suitable formulations include preparations with block-copolymers having adjuvant qualities capable of stimulating the immune response to included antigens, in this case the preferred eotaxin-specific toxoid conjugates.

A specific embodiment of the present invention includes the formulation of the eotaxin-specific toxoid conjugates into the aqueous phase of water-in-oil emulsions. A sterile-filtered (0.1 to 0.2 μm) aqueous solution of the eotaxin-specific toxoid conjugates is combined with a suitable sterile-filtered (0.2 μm) oil mixture containing emulsifiers sufficient to provide for a stable water-in-oil emulsion upon homogenization of the water oil mixture. The emulsification process is practiced as an aseptic procedure within a laminar flow hood or suitable sterile isolator useful for the practice of aseptic formulation and filling of pharmaceutical formulations, including sterile emulsions, that cannot otherwise be sterilized by terminal filtration, heat sterilization or irradiation.

The water-to-oil mixture may be varied in the range 50:50 to 10:90, but more preferably in the range 40:60 to 20:80 water-to-oil. Suitable oil/emulsifier mixtures for this purpose of producing the preferred water-in-oil emulsion may be obtained from the Montanide product range available from SEPPIC, SA, Paris, France. In addition, it may be desirable to further incorporate during the emulsification process a water-soluble adjuvant into the aqueous phase of the final emulsion (Adams, A. Synthetic Adjuvants. 1985 John Wiley & Sons, New York). Examples of suitable adjuvants include, Quill A, QS21, or muramyl dipepetide (nor-MDP).

Anti-eotaxin DNA Vaccines

In an alternative embodiment of the invention DNA constructs comprising nucleic acid sequences encoding the eotaxin peptide fragments described above and further comprising nucleic acid sequences encoding T helper cell epitopes are used as DNA vaccines. Methods of constructing, formulating and administering such DNA vaccines are known in the art and adapted to the eotaxin peptide epitopes of the invention, see WO 00/65058, WO 98/31398, Donnelly et al., 1997, Annu. Rev. Immunol. 15: 617-648 and Donnelly et al., 1997, Life Sciences 60: 163-172.

Incorporation by Reference

Throughout this application reference is made to various publications and patent documents. The disclosures of each of these references is hereby incorporated by reference into this disclosure in its entirety as part of the description of this application as are the disclosures of the publications and patents that are in turn cited therein.

1. A method for treating a subject for a condition mediated by eotaxin, which comprises generating an active immune response in the subject to eotaxin.

2. The method of claim 1 wherein the condition mediated by eotaxin is asthma, allergy or allergic disease.

3. The method of claim 1 wherein the active immune response comprises generating autoantibodies in the subject which bind to eotaxin at a level sufficient to neutralize the effect of eotaxin in mediating the condition.

4. The method of claim 1, which comprises immunizing the subject with an immunogenic composition, which generates antibodies in the subject, which bind to eotaxin.

5. The method of claim 4 wherein the immunogenic composition comprises eotaxin or a portion of eotaxin bound to an immunogenic carrier.

6. The method of claim 5 wherein the immunogenic carrier comprises a T-cell epitope and an eotaxin derived epitope.

7. The method of claim 6 wherein the immunogenic composition comprises an immunogenic analog of the eotaxin.

8. The method of claim 4 wherein the immunogenic composition is a DNA vaccine.

9. An immunogenic composition comprising eotaxin or a peptide fragment thereof coupled to an immunogenic protein carrier.

10. The immunogenic composition of claim 9 comprising a T-cell epitope and an epitope derived from eotaxin.

11. The immunogenic composition of claim 9 comprising eotaxin or a portion thereof conjugated to an immunogenic carrier derived from DT, TT or KLH.

12. A method of producing the composition of claim 9 or 11, which comprises conjugating the eotaxin or a portion thereof to the immunogenic carrier.

13. A pharmaceutical formulation for use as a therapeutic vaccine comprising the immunogenic composition of claim 9, a pharmaceutically acceptable adjuvant and a pharmaceutically acceptable vehicle.

14. The immunogenic composition of claim 9 comprising a peptide sequence selected from the peptide sequences set forth in SEQ ID Nos. 1-38 and 42-61 or mixtures thereof.

15. A DNA vaccine that encodes a peptide, which generates an immune response to eotaxin.


SEQUENCE LISTING (61 sequences)

SEQ ID NO: 1 - protein, 74 aa, Homo sapiens
GPASVPTTCCFNLANRKIPLQRLESYRRITSGKCPQKAVIFKTKLAKDICADPKKKWVQDSMKYLDQKSPTPKP

SEQ ID NO: 2 - protein, 6 aa, Artificial
  Eotaxin epitope
GPASVP

SEQ ID NO: 3 - protein, 7 aa, Artificial
  Eotaxin epitope
GPASVPT

SEQ ID NO: 4 - protein, 8 aa, Artificial
  Eotaxin epitope
GPASVPTT

SEQ ID NO: 5 - protein, 9 aa, Artificial
  Eotaxin epitope
GPASVPTTC

SEQ ID NO: 6 - protein, 10 aa, Artificial
  Eotaxin epitope
GPASVPTTCC

SEQ ID NO: 7 - protein, 11 aa, Artificial
  Eotaxin epitope
GPASVPTTCCF

SEQ ID NO: 8 - protein, 12 aa, Artificial
  Eotaxin epitope
GPASVPTTCCFN

SEQ ID NO: 9 - protein, 13 aa, Artificial
  Eotaxin epitope
GPASVPTTCCFNL

SEQ ID NO: 10 - protein, 14 aa, Artificial
  Eotaxin epitope
GPASVPTTCCFNLA

SEQ ID NO: 11 - protein, 15 aa, Artificial
  Eotaxin epitope
GPASVPTTCCFNLAN

SEQ ID NO: 12 - protein, 16 aa, Artificial
  Eotaxin epitope
GPASVPTTCCFNLANR

SEQ ID NO: 13 - protein, 17 aa, Artificial
  Eotaxin epitope
GPASVPTTCCFNLANRK

SEQ ID NO: 14 - protein, 18 aa, Artificial
  Eotaxin epitope
GPASVPTTCCFNLANRKI

SEQ ID NO: 15 - protein, 19 aa, Artificial
  Eotaxin epitope
GPASVPTTCCFNLANRKIP

SEQ ID NO: 16 - protein, 20 aa, Artificial
  Eotaxin epitope
GPASVPTTCCFNLANRKIPL

SEQ ID NO: 17 - protein, 21 aa, Artificial
  Eotaxin epitope
GPASVPTTCCFNLANRKIPLQ

SEQ ID NO: 18 - protein, 6 aa, Artificial
  Eotaxin epitope
FNLANR

SEQ ID NO: 19 - protein, 7 aa, Artificial
  Eotaxin epitope
FNLANRK

SEQ ID NO: 20 - protein, 8 aa, Artificial
  Eotaxin epitope
FNLANRKI

SEQ ID NO: 21 - protein, 9 aa, Artificial
  Eotaxin epitope
FNLANRKIP

SEQ ID NO: 22 - protein, 10 aa, Artificial
  Eotaxin epitope
FNLANRKIPL

SEQ ID NO: 23 - protein, 21 aa, Artificial
  Eotaxin epitope
KKKWVQDSMKYLDQKSPTPKP

SEQ ID NO: 24 - protein, 20 aa, Artificial
  Eotaxin epitope
KKWVQDSMKYLDQKSPTPKP

SEQ ID NO: 25 - protein, 19 aa, Artificial
  Eotaxin epitope
KWVQDSMKYLDQKSPTPKP

SEQ ID NO: 26 - protein, 18 aa, Artificial
  Eotaxin epitope
WVQDSMKYLDQKSPTPKP

SEQ ID NO: 27 - protein, 17 aa, Artificial
  Eotaxin epitope
VQDSMKYLDQKSPTPKP

SEQ ID NO: 28 - protein, 16 aa, Artificial
  Eotaxin epitope
QDSMKYLDQKSPTPKP

SEQ ID NO: 29 - protein, 15 aa, Artificial
  Eotaxin epitope
DSMKYLDQKSPTPKP

SEQ ID NO: 30 - protein, 14 aa, Artificial
  Eotaxin epitope
SMKYLDQKSPTPKP

SEQ ID NO: 31 - protein, 13 aa, Artificial
  Eotaxin epitope
MKYLDQKSPTPKP

SEQ ID NO: 32 - protein, 12 aa, Artificial
  Eotaxin epitope
KYLDQKSPTPKP

SEQ ID NO: 33 - protein, 11 aa, Artificial
  Eotaxin epitope
YDLQKSPTPKP

SEQ ID NO: 34 - protein, 10 aa, Artificial
  Eotaxin epitope
LDQKSPTPKP

SEQ ID NO: 35 - protein, 9 aa, Artificial
  Eotaxin epitope
DQKSPTPKP

SEQ ID NO: 36 - protein, 8 aa, Artificial
  Eotaxin epitope
QKSPTPKP

SEQ ID NO: 37 - protein, 7 aa, Artificial
  Eotaxin epitope
KSPTPKP

SEQ ID NO: 38 - protein, 6 aa, Artificial
  Eotaxin epitope
SPTPKP

SEQ ID NO: 39 - protein, 7 aa, Artificial
  Spacer peptide
SSPPPPC

SEQ ID NO: 40 - protein, 6 aa, Artificial
  Spacer Peptide
RPPPPC

SEQ ID NO: 41 - protein, 6 aa, Artificial
  Spacer peptide
LPPPPC

SEQ ID NO: 42 - protein, 28 aa, Artificial
  Spacer/Eotaxin epitope
SSPPPPCKKKWVQDSMKYLDQKSPTPKP

SEQ ID NO: 43 - protein, 28 aa, Artificial
  Eotaxin epitope/Spacer
KKKWVQDSMKYLDQKSPTPKPSSPPPPC

SEQ ID NO: 44 - protein, 28 aa, Artificial
  Spacer/Eotaxin epitope
CPPPPSSKKKWVQDSMKYLDQKSPTPKP

SEQ ID NO: 45 - protein, 28 aa, Artificial
  Eotaxin epitope/Spacer
KKKWVQDSMKYLDQKSPTPKPCPPPPSS

SEQ ID NO: 46 - protein, 27 aa, Artificial
  Eotaxin epitope/Spacer
GPASVPTTCCFNLANRKIPLSSPPPPC

SEQ ID NO: 47 - protein, 27 aa, Artificial
  Spacer/Eotaxin epitope
SSPPPPCGPASVPTTCCFNLANRKIPL

SEQ ID NO: 48 - protein, 27 aa, Artificial
  Spacer/Eotaxin epitope
CPPPPSSGPASVPTTCCFNLANRKIPL

SEQ ID NO: 49 - protein, 27 aa, Artificial
  Eotaxin epitope/Spacer
GPASVPTTCCFNLANRKIPLSSPPPPC

SEQ ID NO: 50 - protein, 17 aa, Artificial
  Eotaxin epitope/Spacer
SGKCPQKAVISSPPPPC

SEQ ID NO: 51 - protein, 17 aa, Artificial
  Spacer/Eotaxin epitope
CPPPPSSSGKCPQKAVI

SEQ ID NO: 52 - protein, 17 aa, Artificial
  Eotaxin epitope/Spacer
FKTKLAKDICSSPPPPC

SEQ ID NO: 53 - protein, 17 aa, Artificial
  Spacer/Eotaxin epitope
CPPPPSSFKTKLAKDIC

SEQ ID NO: 54 - protein, 17 aa, Artificial
  Eotaxin epitope/Spacer
ADPKKKWVQDSSPPPPC

SEQ ID NO: 55 - protein, 17 aa, Artificial
  Spacer/Eotaxin epitope
CPPPPSSADPKKKWVQD

SEQ ID NO: 56 - protein, 17 aa, Artificial
  Eotaxin epitope/Spacer
SGKTPQKAVISSPPPPC

SEQ ID NO: 57 - protein, 17 aa, Artificial
  Spacer/Eotaxin epitope
CPPPPSSSGKTPQKAVI

SEQ ID NO: 58 - protein, 17 aa, Artificial
  Eotaxin epitope/Spacer
FKTKLAKDITSSPPPPC

SEQ ID NO: 59 - protein, 17 aa, Artificial
  Spacer/Eotaxin epitope
CPPPPSSFKTKLAKDIT

SEQ ID NO: 60 - protein, 17 aa, Artificial
  Eotaxin epitope/Spacer
ADPKKKWVQDSSPPPPC

SEQ ID NO: 61 - protein, 17 aa, Artificial
  Spacer/Eotaxin epitope
CPPPPSSADPKKKWVQD